(19) United States
(12) Patent Application Publication
(10) Pub. No.: US 2006/0122574 A1
(43) Pub. Date: Jun. 8, 2006

(54) Multi-panel dispenser napkin

(75) Inventor: Chester W. Gooding JR., Neenah, WI (US)

Correspondence Address: PATENT GROUP GA030-43;GEORGIA-PACIFIC CORPORATION, 133 PEACHTREE STREET, N.E., ATLANTA, GA, 30303-1847, US

(73) Assignee: Fort James Corporation, Atlanta, GA (US)

(21) Appl. No.: 11/004,323

(22) Filed: Dec. 3, 2004

(51) Int. Cl. A61F 13/15

(52) U.S. Cl. 604/385.201; 604/385.02

(57) ABSTRACT

A multi-panel dispenser napkin folded on a napkin folder and produced from a paper web and having a plurality of equally sized panels. The width-to-length ratio of the fold configuration is selected from the range of about 0.4 to about 0.7. The fold configuration has at least one transverse fold formed on the folder along the transverse dimension of the paper web and at least two longitudinal folds formed on the napkin folder along the longitudinal dimension of the paper web. None of the equally sized panels has an off-fold panel. The fold configuration is characterized by generally uniform thickness.

### FIELD OF THE INVENTION

This invention relates generally to a multi-panel folded napkin of uniform compressibility and method for loading a napkin dispenser with a stack of multi-panel napkins. More particularly, the invention relates to a stack of multi-panel napkins and a method for folding the napkins to facilitate transportation or storage, as well as to make it easier to remove the folded napkins from the dispenser, particularly when used with an automated dispenser.

### BACKGROUND OF THE INVENTION

Dispensing paper napkins one-at-a-time, reliably, conveniently and quickly presents a considerably more difficult technical challenge than would be readily apparent. Simple dispensers of the type presently encountered in many quick service restaurants are often quite wasteful as it is typically simpler for the customer to remove several napkins at once than it is to remove a single napkin, even if the customer only desires one napkin. Further, many automated dispensers are subject to jamming, failure to feed and multiple feed problems. Dispenser napkins are paper products that are folded in a variety of ways to achieve a defined size, strength and bulk desired for transportation and end use. As used in the art, “conventional dispenser napkin” is used to refer to a napkin (i) in one of the following unfolded sizes: 13″×13″, 12″×12.25″, 15″×15″, 15″×17″, 13″×17″, 12″×17″, 11″×17″, 15″×15″, 15″×17″ or 10″×1″; (ii) having an off-fold configuration; and (iii) having a relatively uniform emboss pattern over its surface. As various kinds of eating establishments become more prevalent, sanitary, and economical, so rapidly dispensable napkins and systems for dispensing them have become highly desirable. Conventional dispenser napkins are found on countertops and tabletops in lunchrooms and restaurants. It is often the case that many conventional dispenser napkins exhibit undesirable tearing or tabbing during the dispensing process.

A variety of napkin configurations can be used to produce a folded dispenser napkin. For example, U.S. Pat. Nos. 2,602,013 and 5,368,188 disclose paper products folded in the “Z-fold” configuration. The Z-fold configuration is not typically used for dispenser napkins.

Another example of a napkin fold configuration is described in U.S. Pat. No. 6,306,480, assigned to the assignee of the present invention. The description relates to an elongate single-ply paper napkin that exhibits sufficient absorbency, strength and hand feel by having the folds running only in the cross-machine direction and being free of longitudinal folds.

As mentioned, conventional dispenser napkins normally have folds in both the longitudinal direction (parallel to the machine direction) and the transverse direction (parallel to the cross machine direction) in an off-fold configuration providing an area for grasping the napkin to pull it out of a dispenser. While the longitudinal and transverse folds make it possible to dispense napkins with a relatively large unfolded area from a relatively compact dispenser, the off-fold causes a difference in bulk across the folded napkin, as well as a soft or more easily compressed area in a stack of such napkins. The difference in softness of a stack often causes crushing and packaging problems in transportation as well as stacking problems in the napkin dispensers. Moreover, the difference in the sizes of the panels of the napkins with both longitudinal and transverse folds in the off-fold configuration causes such napkins, in the folded state, to have a non-uniform thickness and high and low strength areas, often causing a problem during dispensing. When the napkins are stacked or arrayed for insertion into the dispenser, the non-uniform soft, more easily compressed, areas in a napkin stack are disposed in registry with each other, and the non-uniform firm areas in the napkins in the same stack are similarly disposed in registry with each other, creating a firm end and a soft end in the stack. Furthermore, when stacked for shipping, the soft end of the napkin stack may become crushed, deformed or folded, forming wrinkles that interfere with the dispensability of an individual napkin. For example, more than one napkin can be dispensed to a user, increasing the frequency with which a dispenser must be refilled. Since quick service restaurants operate on razor thin margins, both the cost of the wasted napkins and the labor for filling the dispenser are considered significant by the operators.

It is therefore desirable to provide an improved dispenser napkin having an unfolded area equivalent to dispenser size napkins in a more readily dispensed fold configuration which can be manufactured at high speed as well as a method for folding and stacking the napkins for shipping and insertion into a dispenser, while significantly reducing the soft areas within the stack.

### SUMMARY OF THE INVENTION

The present invention is a book folded rectangular napkin having an unfolded size equivalent to conventional dispenser napkins produced of a paper web having a longitudinal dimension and a transverse dimension and folded on a napkin folder having a relatively uniform overall emboss, often bearing an embossed logo but without a coin edge emboss of the type that would cause a difference in the compressibility of the various regions of the stack. The napkin has a plurality of substantially equally sized panels forming a fold configuration of the napkin. The fold configuration has a length and a width with the width-to-length ratio of the fold configuration selected from the range of about 0.4 to about 0.8. The fold configuration has at least one longitudinal, or machine direction, fold formed on the napkin folder and at least two transverse folds formed on the napkin folder. None of the equally sized panels is an off-fold panel and the overall fold configuration is characterized by a generally uniform thickness and compressibility.

A napkin dispenser pack of the present invention comprises a plurality of dispenser napkins, each dispenser napkin having a transverse dimension, a longitudinal dimension and a configuration fold of a generally uniform thickness and compressibility. The fold configuration has at least one longitudinal fold and at least two transverse folds formed on the napkin folder and disposed along the longitudinal dimension. The area of the fold configuration is about the same as a cross-sectional area of the napkin dispenser.

The present invention also provides a method of folding a rectangular dispenser napkin into the desired fold configuration on a folding machine by:
optionally embossing the sheet and slitting it to the desired width, if so required, then;
forming the longitudinal fold or folds using a forming plow;
passing the longitudinally folded web onto a rotating anvil roll which is a combined cutting and vacuum folding roll;
cutting the web to napkin length while the free end, or leading edge, of the web is retained on the rotating anvil roll;
releasing the leading edge of the napkin from the anvil roll and attaching a medial portion to a rotating quarter fold roll;
forming a proximal transverse fold on the rotating quarter fold roll, thereby forming a partially folded napkin;
releasing the partially formed napkin from the quarter fold roll and attaching a portion of the napkins based behind said proximal transverse fold to a packer roll;
forming a distal transverse fold on a packer roll to form a folded napkin having six panels of substantially equivalent size having a spine edge presenting no free sheet edge and a fly-leaf edge presenting an opening;
releasing the folded napkin from the packer roll; and
stacking the folded napkin on a conveyor to ultimately form a dispenser package having the spine edges arrayed along a single edge of the dispenser package.

The fold configuration of each napkin in the dispenser package is of a generally uniform thickness and compressibility and has at least two transverse folds and at least one longitudinal fold. The resulting fold configuration has a plurality of substantially equally sized panels with no substantial off-fold panels with each panel preferably having a width-to-length ratio in the range of about 0.4 to about 0.8.

According to the present invention, a method of loading a napkin dispenser calls for obtaining a plurality of multi-panel napkins with a fold configuration having a plurality of generally equally sized panels with no substantial off-fold panels. The length and width of each napkin are such that the width-to-length ratio of the folded configuration is selected from the range of about 0.4 to about 0.8.

The napkin dispenser having reloading and dispensing configurations is provided to house a dispenser package of napkins of the present invention. The lateral dimensions of the fold configuration are about the same as the interior cross sectional dimensions of the napkin dispenser. Disposing the plurality of the multi-panel napkins adjacent to each other with each napkin having the same orientation creates a stack of multi-panel napkins in the dispenser while the dispenser is in the reloading configuration. Loading the stack of multi-panel napkins into the napkin dispenser in the dispensing configuration and closing the dispenser makes it ready for service. When used with a metering dispenser having an inter-penetrating dispensing mechanism, the napkins in the array are disposed such that the spine edge of each folded napkin is presented to the inter-penetrating mechanism while the fly-leaf edges are disposed away from the location in which the inter-penetrating mechanism enters the stack of napkins. The above discussed and other features and advantages of the present invention will be appreciated and understood by those skilled in the art from the following detailed description and drawings.

### BRIEF DESCRIPTION OF THE DRAWINGS

Referring to the exemplary drawings wherein like elements are numbered alike in the following Figures:

FIG. 1(a) is a schematic illustration of stage 1 of the folding process.

FIG. 1(b) is a schematic illustration of stage 2 of the folding process.

FIG. 1(c) is a schematic illustration of stage 3 of the folding process.

FIG. 1(d) is a schematic illustration of stage 4 of the folding process.

FIG. 2 is a layout of the folds and panels of a napkin.

FIG. 3(a) is a schematic illustration of the napkin traveling around an anvil roll.

FIG. 3(b) illustrates the napkin of FIG. 2 after folding along the longitudinal fold.

FIG. 4(a) is a schematic illustration of the napkin finishing its travel around the anvil roll.

FIG. 4(b) is an illustration of the cut longitudinally folded napkin as arrayed on the anvil roll.

FIG. 5(a) is a schematic illustration of the napkin on the quarter fold roll as the proximal transverse fold is being formed.

FIG. 5(b) is a schematic illustration of the partially folded napkin after the first fold has been placed in the napkin.

FIG. 6(a) is a schematic illustration of the partially folded napkin continuing its travel around the quarter fold roll.

FIG. 6(b) is an illustration of the napkin with the first fold arrayed around the quarter fold roll.

FIG. 7(a) is a schematic illustration of the napkin finishing its travel around the quarter fold roll.

FIG. 7(b) is an illustration of the napkin with the first fold as it finishes its travel around the quarter fold roll.

FIG. 8(a) is a schematic illustration of the napkin on the packer roll as the distal transverse fold is being formed.

FIG. 8(b) is an illustration of the napkin just after the distal transverse fold has been formed.

FIG. 9(a) is a schematic illustration of the napkin with two transverse folds formed on the packer roll.

FIG. 9(b) is an illustration of the napkin with two completed transverse folds.

FIG. 10 is a schematic perspective illustrating the fully folded configuration of a single napkin.

FIG. 11 is a perspective of a stack of napkins as arrayed for placement into a dispenser having the inter-penetrating metering mechanism.

FIG. 12(a) is a cross-sectional view of a stack of napkins properly arrayed in a dispenser having an inter-penetrating metering mechanism.

FIG. 12(b) is an illustration of a stack of napkins.

FIG. 12(c) is a front elevational view of a stack of napkins in an open dispenser.

### DETAILED DESCRIPTION OF THE INVENTION

The paper web 800 used to produce the multi-panel napkins may be manufactured on any conventional papermaking machine (not shown). The web may be embossed to obtain the required softness and appearance. Typically, this is done just prior to slitting the parent roll down to the required width, but pre-embossed or unembossed rolls may also be used. Unembossed rolls are particularly suitable if the base sheet is formed by through air drying and bears an attractive wire or fabric pattern. Embossing patterns can be any overall emboss pattern, spot emboss pattern, micro emboss pattern, (micro emboss patterns being patterns made of regularly shaped (usually elongate) elements whose long dimension is 0.050 inches or less), or combinations of overall, spot, and micro emboss patterns. The emboss pattern of napkins of the present invention will normally be relatively uniform overall and will not bear a substantial coin-edge or other emboss of the type which would provide marked differences in the compressibility of the one edge of the stack as compared to another although a minimal or very lightly embossed design can be used along the edge so long as the apparent or superficial thickness of the napkin remains substantially uniform over its entire surface.

A paper napkin is folded using a conventional automated folder capable of forming transverse and longitudinal folds, resulting in a “book fold” configuration or a “tablet fold” configuration. Suitable folders are manufactured by C. G. Bretting Manufacturing Co. and are described in the following U.S. Patents:

U.S. Pat. No. 6,547,909, Flying Web Splice Apparatus And Method;

U.S. Pat. No. 6,539,829, Rotary Valve Assembly And Method;

U.S. Pat. No. 6,508,153, Conveyor Product Transfer Apparatus And Method;

U.S. Pat. No. 6,488,194, Vacuum Timing Device And Method For Producing The Same;

U.S. Pat. No. 6,431,038, Vacuum Assisted Method Of Cutting A Web Material;

U.S. Pat. No. 6,372,064, Tail Sealer Apparatus And Method;

U.S. Pat. No. 6,322,315, Web Stacker And Separator Apparatus And Method;

U.S. Pat. No. 6,296,601, Vacuum Assisted Roll Apparatus And Method;

U.S. Pat. No. 6,254,522, Separator Finger Apparatus;

U.S. Pat. No. 6,227,086, Radial Log Clamp;

U.S. Pat. No. 6,138,543, Blade Mounting Arrangement For Cut-Off System;

U.S. Pat. No. 6,051,095, Flying Web Splice Apparatus And Method;

U.S. Pat. No. 6,000,657, Winding Control Finger Surface Rewinder With Core Insert

Finger; U.S. Pat. No. 5,941,144, Radial Log Clamp;

U.S. Pat. No. 5,820,064, Winding Control Finger Surface Rewinder With Core Insert Finger;

U.S. Pat. No. 5,772,149, Winding Control Finger Surface Rewinder;

U.S. Pat. No. 5,755,146, Radial Log Clamp;

U.S. Pat. No. 5,643,398, Log Tail Sealer;

U.S. Pat. No. 5,584,443, Rewinder Log Control;

U.S. Pat. No. 5,299,793, Multi-Panel Refolding Transfer System With Rotating Transfer Clamp;

U.S. Pat. No. 5,226,611, Twin Station Rewinder;

U.S. Pat. No. 4,997,338, Short Count Sheet Separator;

U.S. Pat. No. 4,917,665, Bedroll Interfolding Machinery Improvement;

U.S. Pat. No. 4,874,158, Dispensing Fold Improvement For A Clip Separator;

U.S. Pat. No. 4,778,441, Interfolding Machinery Improvement;

U.S. Pat. No. 4,770,402, Clip Separator For Interfolded Sheets;

U.S. Pat. No. 4,765,604, Resilient Creaser;

U.S. Pat. No. 4,751,807, Automatic Transfer System;

U.S. Pat. No. 4,475,730, Apparatus For Folding And Stacking Paper Products;

U.S. Pat. No. 4,270,744, Tuckers On Mechanical Folding Rolls;

U.S. Pat. No. 4,254,947, Sheet Overlap Device; and

U.S. Pat. No. 3,709,077, Cut-Off Device.

In accordance with an exemplary embodiment, napkins 100 are formed on an automated folding machine combining forming plows or plates 802 for creating longitudinal folds 810 in the longitudinal dimension, i.e. machine direction, of the web and vacuum folding rolls for creating transverse folds in the transverse dimension, or cross-machine direction, of the web. The forming plows or plates 802 and the vacuum folding rolls 809, 824, 840 fold the rectangular napkin 100 into a fold configuration of a generally uniform thickness and compressibility by creating at least one longitudinal fold and at least two transverse folds, wherein the fold configuration has a plurality of generally equally sized panels 816, 818, 820, 822, 825, and 826 having no substantial off-fold panels and being characterized by a length and a width having a width-to-length ratio of the range of about 0.4 to about 1.0.

“Book folded” paper napkins typically include at least two transverse folds. One transverse free edge of the web is folded toward the other transverse free edge to create two panels in the web. The transverse fold in the napkin is then folded in the same direction as the first fold toward the transverse free edge of the web. If more folds are desired, the most recently formed transverse fold in the napkin is folded toward the transverse free edge of the web in the same direction as the previous folds. The first transverse free edge of the web may be folded “up” or “down” toward the other transverse free edge, wherein “up” and “down” are relative terms that may be dependent upon the orientation of the web during the folding process. “Tablet folded” paper napkins typically include an accordion-like fold. Tablet folded napkins are not suitable for the present invention as there are more “wrong” ways to load tablet folded napkins into an automated dispenser than there are with book folded napkins.

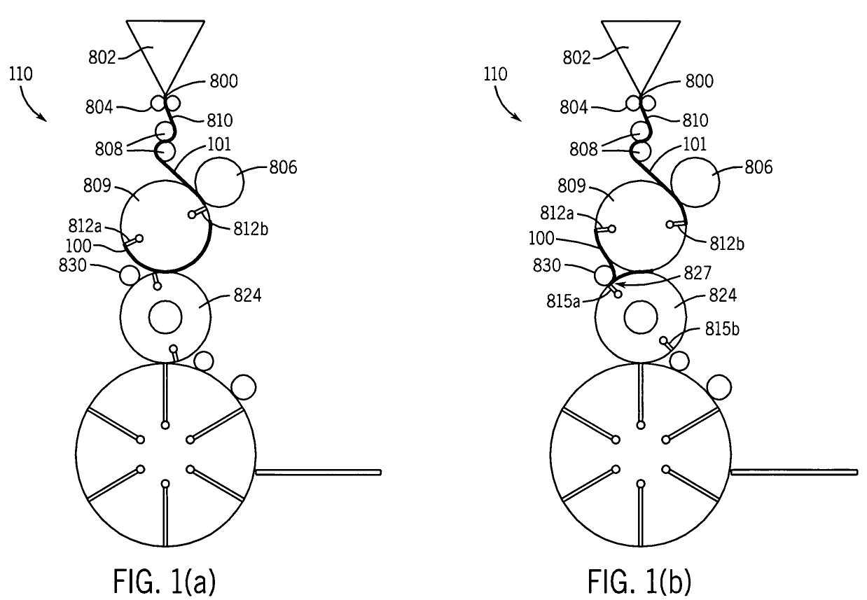

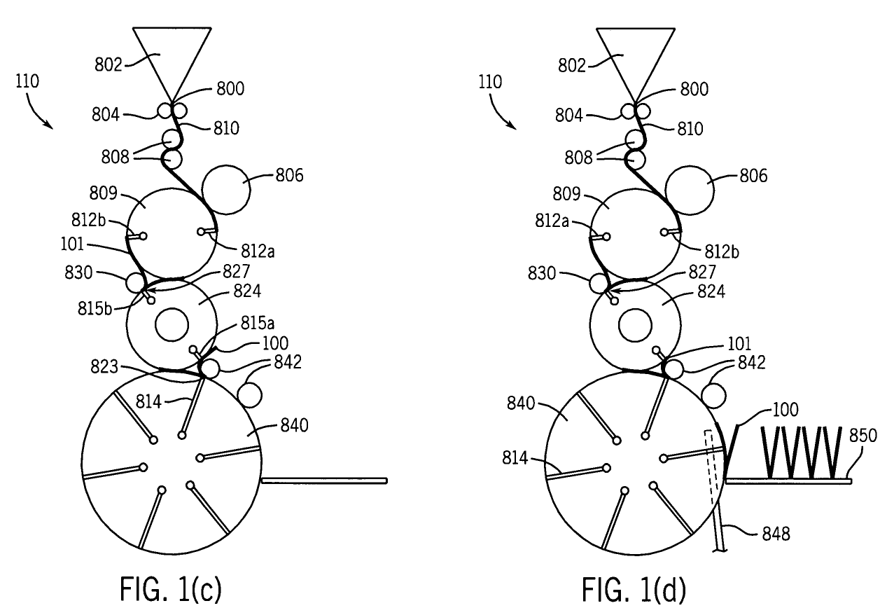

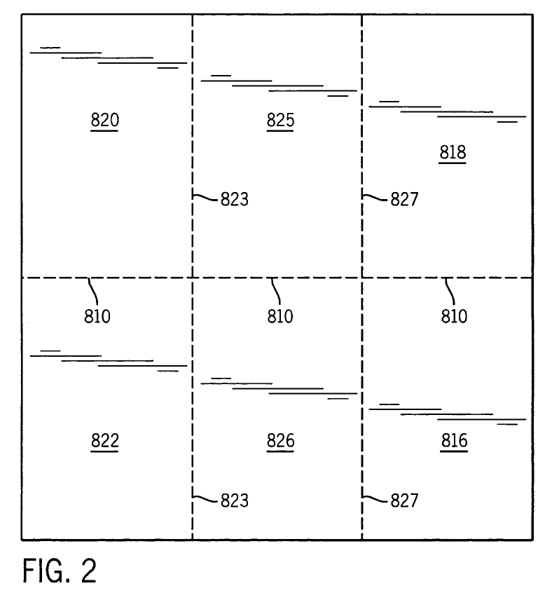

Specifically, according to the present method of folding a six-panel napkin, FIGS. 1(a)-(d) schematically illustrate four stages of folding a napkin 100 on a folding machine 110. In FIG. 2, napkin 100 is illustrated as first folded in accordance with the present invention, then unfolded to better illustrate the overall geometry of the napkin. As seen in FIG. 2, napkin 100 has six equally sized panels 816, 818, 820, 822, 825 and 826, two transverse folds 823 and 827 and one longitudinal fold 810. At stage 1 of the folding process shown in FIG. 1(a), longitudinal fold 810 is impressed upon a paper web 800 as it passes over forming plow 802. Web 800 with longitudinal fold 810 travels through a number of rolls (nose rolls 804 and pull rolls 808) to anvil roll 809. Web 800 is retained on anvil roll 809 by vacuum and travels around anvil roll 809 to fly knife roll 806 where it is cut by fly knife roll 806 to the length of napkin 100.

After being cut to length, napkin 100 is retained by anvil roll 809's first vacuum zone shown as 812a in FIG. 1(a), wherein napkin 100 wraps itself along a portion of the circumference of anvil roll 809. At stage 2, schematically illustrated in FIG. 1(b), the vacuum in vacuum zone 812a of the anvil roll is turned off, while vacuum is applied to zone 815a in quarter fold roll 824 so as to engage napkin 100 approximately one third of its length from its leading edge. Accordingly, napkin 100 leaves anvil roll 809 to travel around quarter fold roll 824 to form proximal transverse fold 827 (shown in FIG. 1(b)) between idler roll 830 and quarter fold roll 824. At stage 3 of the folding process, shown schematically in FIG. 1(c), fold 827 at the leading edge of the subsequent napkin 101 passes around quarter fold roll 824, and under quarter fold idler roll 830. As the trailing edge of the folded portion of napkin 100 contacts packer roll 840, the vacuum is released in first quarter fold vacuum zone 815a which previously retained the folded portion of napkin 100 on quarter fold roll 824 while, simultaneously, vacuum is applied to vacuum zone 814a on packer roll 840 so that napkin 100 leaves the quarter fold roll and is drawn against packer roll 840, whereupon it travels around packer roll 840, passing between packer roll 840 and ironing roll 842 forming distal transverse fold 823. At stage 4, schematically shown in FIG. 1(d), fully folded napkin 100 travels further around packer roll 840 and is then packed off onto a separator table top 850 by packer finger 848.

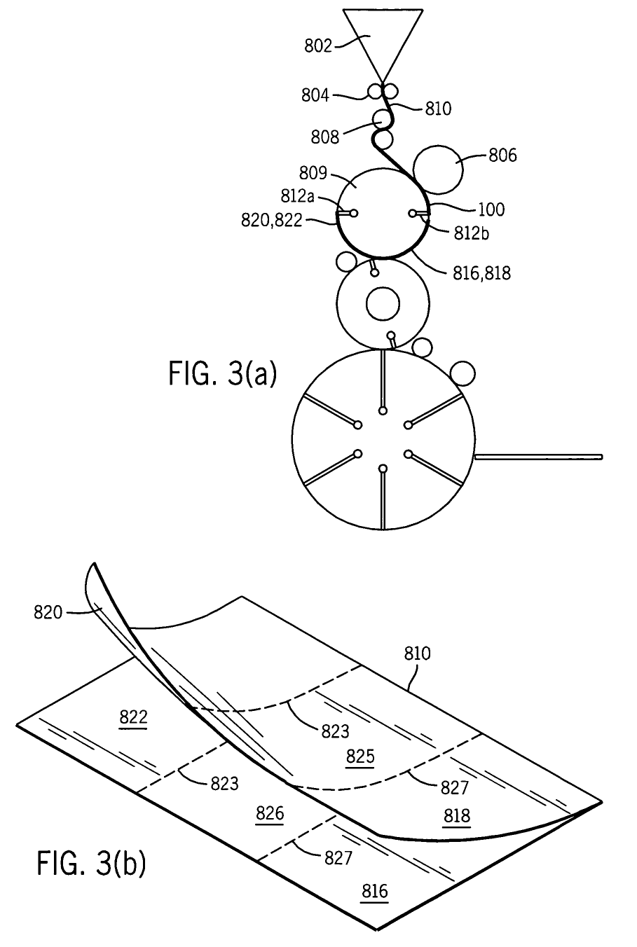

To illustrate the six-panel napkin folding process in more detail, FIG. 3(a) schematically illustrates napkin 100 being folded along longitudinal fold 810 as it passes over forming plow 802 past nose rolls 804, through pull rolls 808, onto anvil roll 809 where it is cut off to size by fly knife roll 806 while traveling along anvil roll 809. Vacuum (representatively shown as two vacuum zones 812a and 812b in anvil roll 809) holds napkin 100 in close contact with anvil roll 809. While on anvil roll 809, napkin 100 is arrayed along anvil roll 809 in a semi-circle much like a portion of the periphery of an oatmeal box. Longitudinal fold 810 divides napkin 100 into upper and lower halves. The upper half consists of panels 820, 825, and 818, as shown in FIG. 3(b), the lower half consists of panels 822 (under panel 820), 826 (under panel 825), and 816 (under panel 818), with panels 822 and 820 located closer to the 9 o'clock position on anvil roll 809 and with panels 816 and 818 located closer to the 4 o'clock position on the anvil roll 809, as shown in FIG. 3(a).

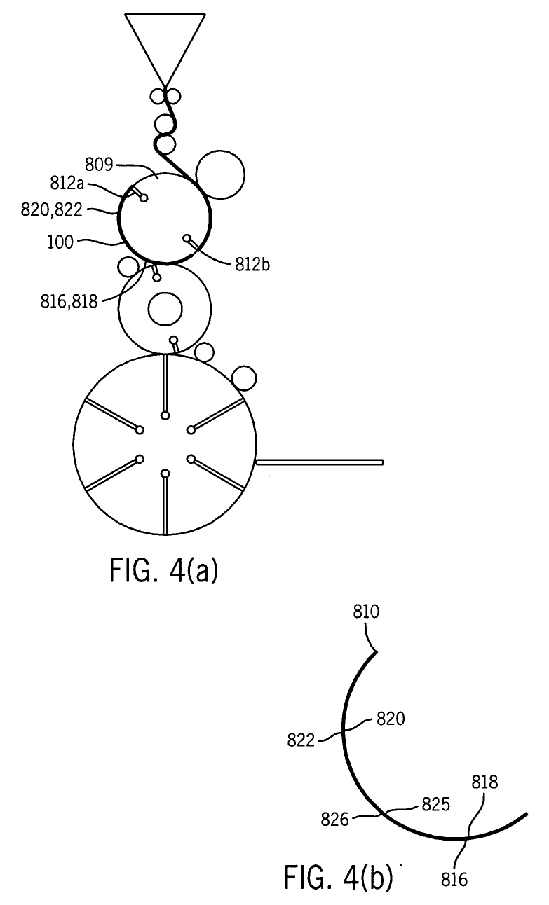

FIG. 4(a) illustrates napkin 100 on anvil roll 809 advancing to about the 11 o'clock position on anvil roll 809 as it rotates while vacuum zone 812a holds napkin 100 firmly to the surface of anvil roll 809. Panels 820 and 822 are located close to the 10 o'clock position on anvil roll 809 in FIG. 4(a), while panels 818 and 816 of napkin 100 are located closer to the 6 o'clock position on the anvil roll. FIG. 4(b) illustrates the position of the napkin on the anvil roll in the rotational position shown in FIG. 4(a).

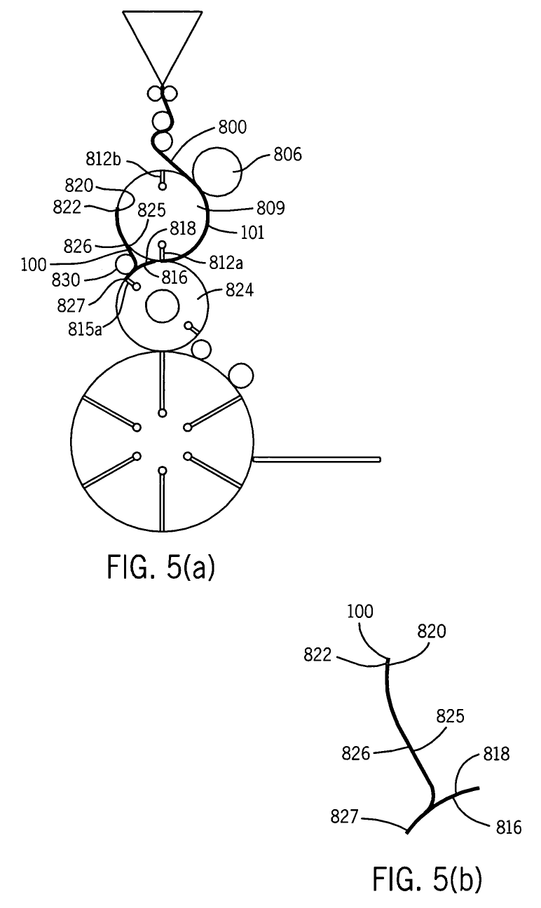

FIG. 5(a) illustrates formation of proximal transverse fold 827 of napkin 100. As the edge of napkin 100 adjacent panels 820 and 822 approaches the 10 o'clock position on anvil roll 809, the vacuum in vacuum zone 812a holding panels 816 and 818 wrapped around anvil roll 809 is shut off, releasing napkin 100 from anvil roll 809. At the same time, vacuum zone 815a located in quarter fold roll 824 is activated and the vacuum in zone 815a pulls panels 818 and 816 (panel 816 bearing against quarter fold roll 824) to the surface of quarter fold roll 824. As panels 818 and 816 are pulled onto quarter fold roll 824 as illustrated in FIG. 5(a), the rest of napkin 100 releases and is drawn away from the surface of anvil roll 809 by vacuum in vacuum zone 815a. As napkin 100 wraps around quarter fold roll 824 after being released from anvil roll 809, it passes between quarter fold roll 824 and idler roll 830 thereby forming proximal transverse fold 827 upon continued rotation of quarter fold roll 824. It should also be noted that while napkin 100 is transferring off of anvil roll 809, wrapping itself around and being folded on quarter fold roll 824, a subsequent napkin 101 is being cut from web 800 to size by fly knife roll 806.

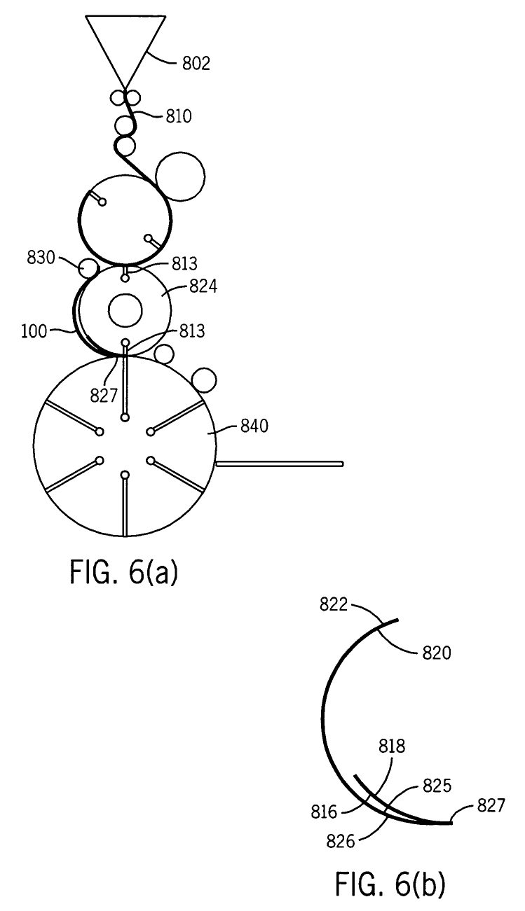

FIG. 6(a) schematically illustrates the position of napkin 100 as quarter fold roll 824 rotates bringing napkin 100 into wrapping engagement around the surface of quarter fold roll 824 with proximal transverse fold 827 disposed as shown in FIG. 6(a) at about the 7 o'clock position on quarter fold roll 824. FIG. 6(b) is a schematic showing the disposition of the various panels 816, 818, 820, 822, 825, 826 of napkin 100 on quarter fold roll 824 just prior to contact with packer roll 840 wherein panels 818 and 816 have been folded against panels 825 and 826 forming proximal transverse fold 827.

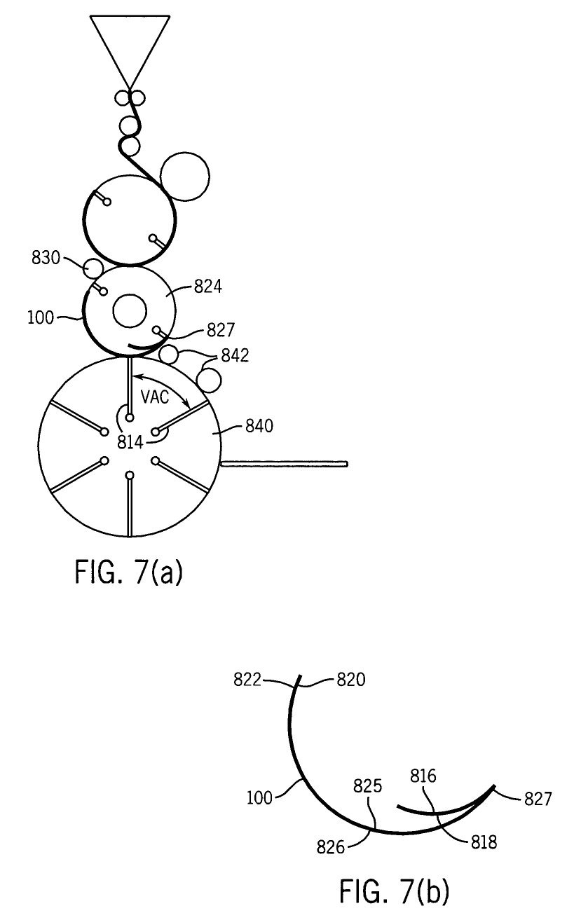

FIG. 7(a) shows the disposition of napkin 100 as it continues to travel around rotating quarter fold roll 824. As napkin 100 wraps around rotating quarter fold roll 824, proximal transverse fold 827 reaches the point between packer ironing roll 842 and quarter fold roll 824. (Note that napkin 100 does not engage packer ironing roll 842 at this time). FIG. 7(b) illustrates the disposition of panels 816, 818, 820, 822, 825, 826 of napkin 100 on quarter fold roll 824 as proximal transverse fold 827 reaches about the 5 o'clock position on quarter fold roll 824. Proximal transverse fold 827 is at the 5 o'clock position with adjacent pairs of panels 818/816 and 825/826 ranging roughly between the 5 o'clock and 7 o'clock positions with panels 820 and 822 being placed between the 7 and 9 o'clock positions as shown in FIG. 7(b).

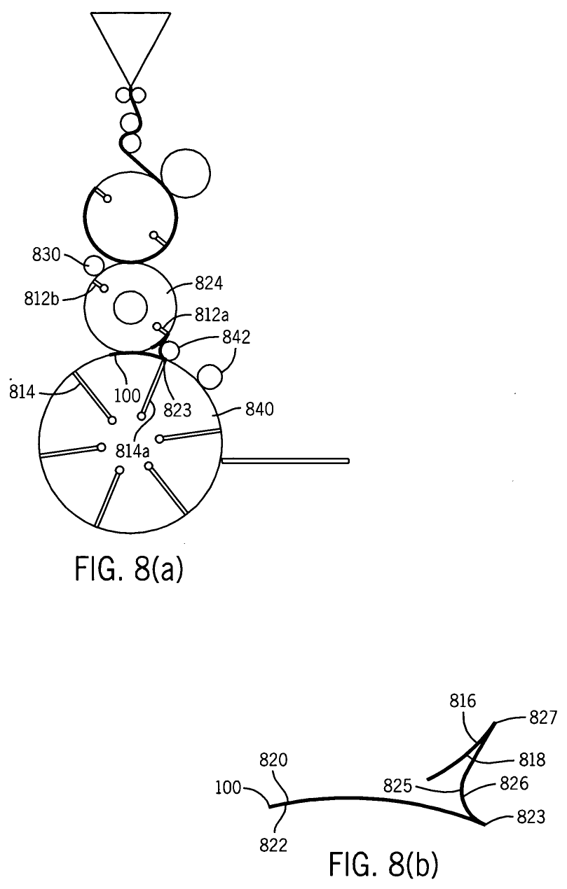

FIG. 8(a) illustrates formation of distal transverse fold 823 in napkin 100. As the trailing edge of panel 816 on quarter fold roll 824 passes the 6 o'clock position on quarter fold roll 824 corresponding to the 12 o'clock position on packer roll 840, vacuum is withdrawn from vacuum zone 812a of quarter fold roll 824 releasing napkin 100 to be picked up by packer roll 840 as vacuum is applied to vacuum zone 814a drawing panels 820/822 onto the surface of packer roll 840 as shown in FIG. 8(b). Medial panels 825 and 826 of napkin 100 as well as proximal panels 816 and 818 are drawn between packer roll 840 and packer roll ironing roll 842, forming distal transverse fold 823. FIG. 8(b) shows the configuration the various panels of napkin 100 as distal fold 823 is formed between packer roll 840 and packer roll ironing roll 842.

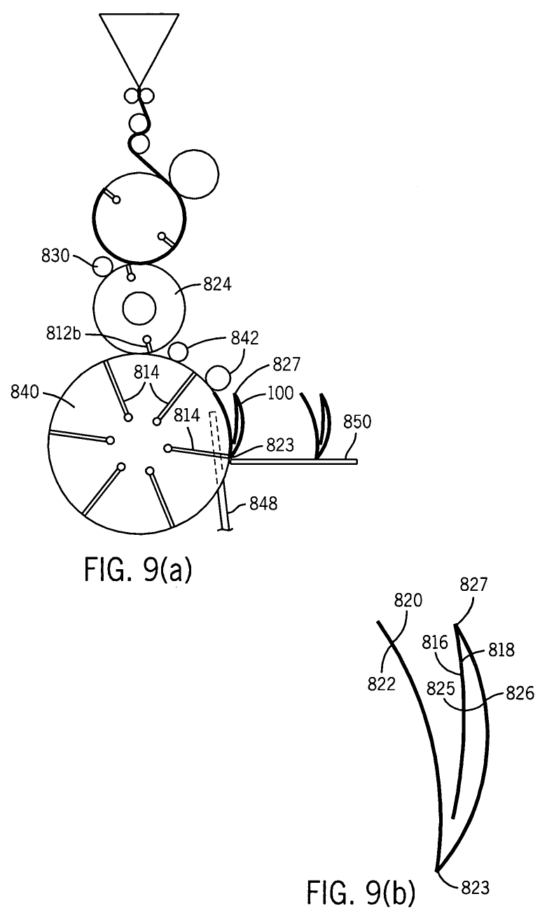

FIG. 9(a) shows napkin 100 with both formed transverse folds 827 and 823 disposed around packer roll 840 as napkin 100 traverses around packer roll 840. The disposition of the various panels in folded napkin 100 is shown in FIG. 9(b), wherein proximal transverse fold 827 and distal transverse fold 823 define three overlaying areas of the napkin: one area comprising panels 820/822; another, panels 825/826; and the third, panels 818/816. As previously described with regard to FIGS. 1(a) and 1(b), folded napkin 100 exhibits one longitudinal fold 810 separating panels 820 and 822 from each other and similarly separating panels 825 from 826 as well as 818 from 816.

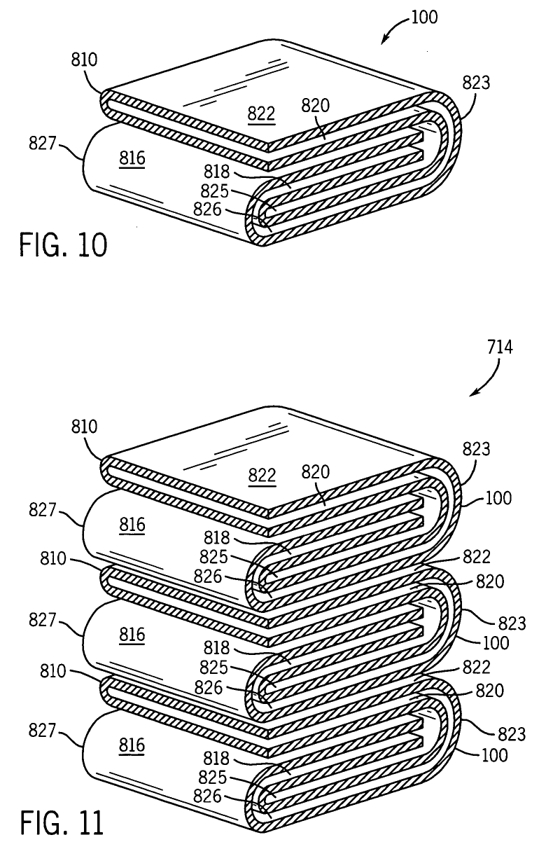

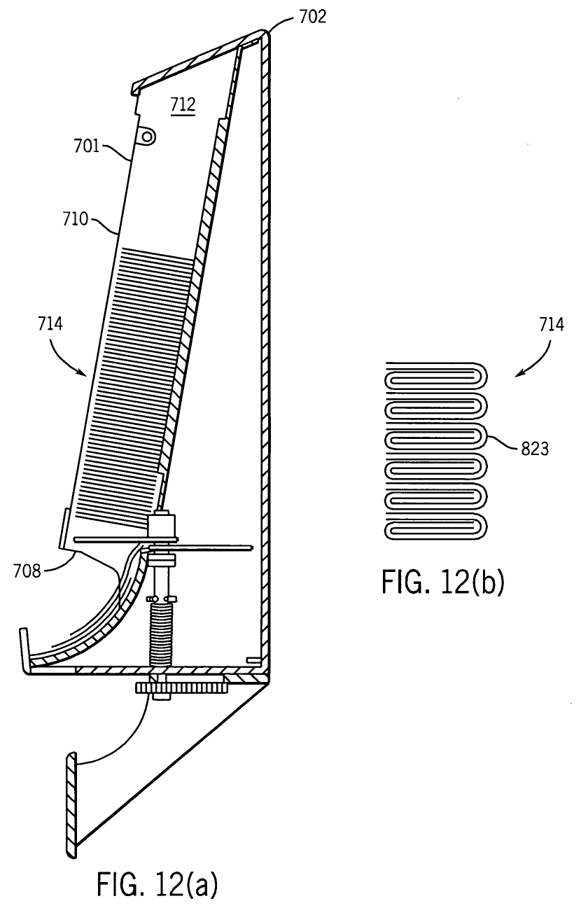

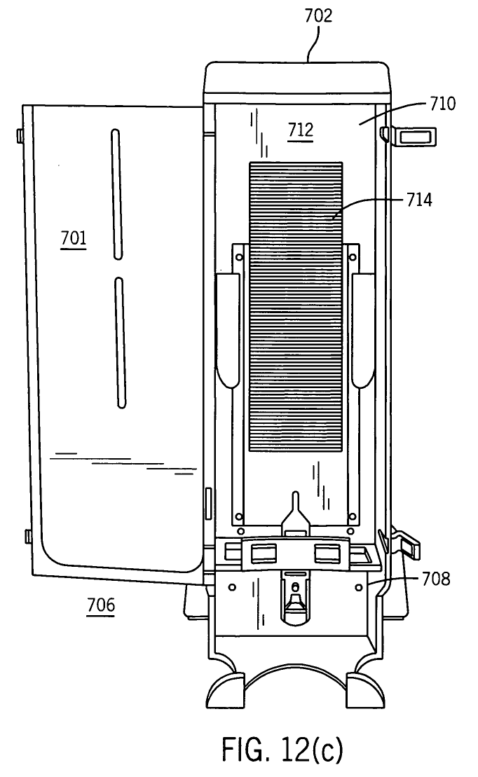

Referring to FIG. 10, one folded multipanel napkin 100 is illustrated and in FIG. 11 a plurality of multi-panel napkins 714 are shown in a stack. It is to be noted that analogous to a book, fold 823 forms a spine edge while the flyleaf edge of the napkin is formed by fold 827 and adjacent panels 816, 820, and 822. As shown in FIG. 12(a) and FIG. 12(c), door 701 on napkin dispenser 702 can be positioned into a dispensing configuration as shown in FIG. 12(a) and a reloading configuration as shown in FIG. 12(c). A dispensing opening 708 and reload opening 710 are coupled with dispenser cavity 712. Preferably, when door 701 of napkin dispenser 702 is in the reloading configuration, napkin dispenser 702 is configured to allow access to dispenser cavity 712 via the reload opening 710. Although napkin dispenser 702 is preferably a metering napkin dispenser, napkin dispenser 702 can be any type of a napkin dispenser. When used with an interpenetrating metering dispenser mechanism, spine edge defined by fold 823 is disposed rightwardly so as to be presented to the interpenetrating mechanism which enters stack 714 from the right to facilitate accurate dispensing.

A plurality 714 of multi-panel napkins 100 shown in FIG. 11 are assembled adjacent to each other to form a stack of multi-panel napkins 714. Door 701 of napkin dispenser 702 in FIG. 12(c) is then positioned to be in reloading configuration 706, and stack of multi-panel napkins 714 is disposed within dispenser cavity 712 via reload opening 710. Once the reload has been completed, door 701 of napkin dispenser 702 is then repositioned to be in dispensing configuration. Preferably, when napkin dispenser 702 is in dispensing configuration, napkin dispenser 702 only allows access to the napkins in dispenser cavity 712 via dispenser opening 708.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated, any use of the terms first, second, etc., do not denote any order or importance, but rather the terms first, second, etc., are used to distinguish one element from another.

What is claimed is:

1. A rectangular book folded dispenser sized napkin folded on a napkin folder and produced of a paper web having an emboss pattern of substantially uniform caliper, a machine direction dimension and a cross machine direction dimension, the dispenser sized napkin comprising:
a plurality of equally sized panels forming a fold configuration of the napkin having substantially uniform compressibility over its area, the fold configuration having a generally uniform thickness, a length, a width, and a width-to-length ratio of the folded configuration selected from the range of about 0.4 to about 0.8; and
the fold configuration comprising at least one machine direction fold of the paper web and at least two cross machine direction folds;
wherein none of the equally sized panels has a substantial off-fold area.

2. The dispenser napkin of claim 1, wherein the number of equally sized panels is six.

3. The dispenser napkin of claim 1, wherein the number of equally sized panels is eight.

4. The dispenser napkin of claim 2, wherein the width-to-length ratio of the folded configuration is from about 0.5 to about 1.0.

5. The dispenser napkin of claim 3, wherein the width-to-length ratio of the folded configuration is from about 0.4 to about 0.8.

6. The dispenser napkin of claim 3, wherein the width-to-length ratio of the folded configuration is about 0.65.

7. The dispenser napkin of claim 1, wherein the uniform thickness of the fold configuration is from about 25 mils to about 90 mils.

8. The dispenser napkin of claim 1, wherein the plurality of equally sized panels comprise an embossing pattern.

9. A napkin dispenser system comprising:
a housing;
a stack of dispenser napkins comprising a plurality of bookfolded dispenser napkins disposed within said housing, each dispenser napkin having a transverse dimension, a longitudinal dimension and being disposed in a fold configuration exhibiting generally uniform thickness; and
the fold configuration comprising at least one longitudinal fold disposed along the machine direction and at least two transverse folds disposed along the cross-machine direction, the configuration fold comprising a plurality of equally sized panels having a length and a width, and wherein a width-to-length ratio of the folded configuration is selected from the range of about 0.4 to about 1.0;
wherein none of the plurality of the equally sized panels is an off-fold panel and wherein an area of the fold configuration is about the same as a cross sectional area of the napkin dispenser housing; the bookfold in each napkin in the stack of napkins being arranged on a single edge of the stack adjacent the bookfold in the adjacent napkins in the stack; and
an interpenetrating mechanism adapted to enter the stack between the bookfolds in adjacent napkins in the stack.

10. The napkin dispenser of claim 9, wherein the napkin dispenser is mechanically operated or electronically operated.

11. The napkin dispenser of claim 9, wherein a number of the plurality of equally sized napkin panels is six.

12. The napkin dispenser of claim 9, wherein a number of the plurality of equally sized napkin panels is eight.

13. The napkin dispenser of claim 11, wherein the width-to-length ratio of the folded configuration is from about 0.5 to about 1.0.

14. The napkin dispenser of claim 12, wherein the width-to-length ratio of the folded configuration is from about 0.4 to about 0.8.

15. The napkin dispenser of claim 12, wherein the width-to-length ratio of the folded configuration is about 0.65.

16. The napkin dispenser of claim 9, wherein the uniform thickness of the folded configuration is from about 25 mils to about 90 mils.

17. The napkin dispenser of claim 9, wherein the plurality of equally sized panels comprise an embossing pattern.

18. The napkin dispenser of claim 9, further alternately configurable from a reloading configuration to a dispensing configuration.

19. A method of folding a rectangular dispenser napkin into a folded configuration, the method comprising:
providing a folding machine comprising forming plates for creating longitudinal folds in a longitudinal dimension of the machine and controlled vacuum retention turning rolls for creating transverse folds in a transverse dimension of the machine;
feeding a continuous paper web into the folding machine;
cutting the paper web on an anvil roll to provide a rectangular napkin sheet; and
passing the cut napkin to a quarter fold roll and forming a first transverse fold thereupon;
passing the napkin to a packer roll and forming a second transverse fold thereupon;
wherein the forming plates and the controlled vacuum retention turning rolls are configured to fold the rectangular napkin sheet into a fold configuration of a generally uniform thickness by creating at least one longitudinal fold disposed along the machine direction and at least two transverse folds disposed along the cross machine direction, the fold configuration comprising a plurality of equally sized panels with no off-fold panels and having a length and a width defining a width-to-length ratio, the width-to-length ratio of the folded configuration being selected from the range of about 0.4 to about 1.0.

20. The method of claim 19, wherein the number of the plurality of equally sized panels is six or eight.

21. The method of claim 20, wherein the width-to-length ratio of the folded configuration is from about 0.5 to about 1.0.

22. The method of claim 20, wherein the width-to-length ratio of the folded configuration is from about 0.4 to about 0.5.

23. The method of claim 20, wherein the width-to-length ratio of the folded configuration is about 0.65.

24. The method of claim 20, wherein the uniform thickness of the folded configuration is about 25 mils to about 90 mils.

25. The method of claim 20, wherein the plurality of equally sized panels comprise an embossing pattern.

26. A method of loading a napkin dispenser having an interpenetrating dispenser mechanism comprising:
obtaining a plurality of bookfolded multi-panel napkins, wherein each bookfolded multi-panel napkin comprises a fold configuration with a plurality of equally sized panels, none of which is an off-fold panel, the fold configuration having a length and a width defining a width-to-length ratio of the folded configuration from the range of about 0.4 to about 1.0;
obtaining a napkin dispenser having a reloading configuration and a dispensing configuration, and having a cross-sectional area of about the same size as that of the fold configuration;
disposing the plurality of bookfolded multi-panel napkins adjacent to each other to form a stack of multi-panel napkins having said bookfolds aligned along the same face of the stack of bookfolded multi-panel napkins;
positioning the napkin dispenser to be in the reloading configuration;
loading the stack of bookfolded multi-panel napkins into the napkin dispenser such that the bookfolded face of said stack of bookfolded multi-panel dispenser napkins is presented to said interpenetrating mechanism; and
re-positioning the napkin dispenser to be in the dispensing configuration.

27. The method of claim 26, wherein each multi-panel napkin comprise a plurality of equal size napkin panels.

28. The method of claim 26, wherein the napkin dispenser further comprises a dispenser cavity, a reload opening and a dispenser opening, the reload opening and dispenser opening being coupled with the dispenser cavity.

29. The method of claim 28, further comprising accessing the dispenser cavity via the reload opening when the napkin dispenser is in the reloading configuration.

30. A stack of dispenser napkins comprising:
a wrap;
a plurality of bookfolded dispenser napkins encompassed by said wrap, each dispenser napkin having a transverse dimension, a longitudinal dimension and being disposed in a fold configuration exhibiting generally uniform thickness; and
the fold configuration comprising at least one longitudinal fold disposed along the machine direction and at least two transverse folds disposed along the cross-machine direction, the configuration fold comprising a plurality of equally sized panels having a length and a width, and wherein a width-to-length ratio of the folded configuration is selected from the range of about 0.4 to about 1.0;
wherein none of the plurality of the equally sized panels is an off-fold panel and wherein an area of the fold configuration is about the same as a cross sectional area of the napkin dispenser housing; the bookfold in each napkin in the stack of napkins being arranged on a single edge of the stack adjacent the bookfold in the adjacent napkins in the stack.
